(19) United States
(12) Patent Application Publication
(10) Pub. No.: US 2021/0387952 A1
(43) Pub. Date: Dec. 16, 2021

(54) SOLID STATE FORMS OF DAPRODUSTAT AND PROCESS FOR PREPARATION THEREOF

(71) Applicant: TEVA PHARMACEUTICALS INTERNATIONAL GMBH, Jona (CH)

(72) Inventors: Dijana S. Samec, Jastrebarsko (HR); Ivana Landeka, Zagreb (HR); Valentina Travancic, Zagreb (HR)

(21) Appl. No.: 17/291,312

(22) PCT Filed: Nov. 13, 2019
(86) PCT No.: PCT/US19/61119

§ 371 Date: May 5, 2021

Related U.S. Application Data
(60) Provisional application No. 62/835,089, filed on Apr. 17, 2019.
(60) Provisional application No. 62/805,405, filed on Feb. 14, 2019.
(60) Provisional application No. 62/767,580, filed on Nov. 15, 2018.

(51) Int. Cl. C07D 239/60

(52) U.S. Cl. CPC C07D 239/60; C07B 2200/13

(57) ABSTRACT

The present disclosure encompasses solid state forms of Daprodustat, particularly crystalline polymorphs of Daprodustat, processes for preparation thereof, and pharmaceutical compositions thereof.

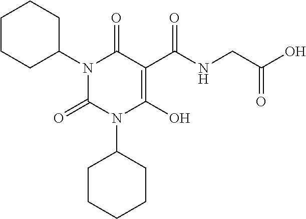 [SMILES: O=C(O)CNC(=O)c1c(O)n(C2CCCCC2)c(=O)n(C2CCCCC2)c1=O]

### FIELD OF THE DISCLOSURE

The present disclosure encompasses solid state forms of Daprodustat, particularly crystalline polymorphs of Daprodustat, processes for preparation thereof, and pharmaceutical compositions thereof.

### BACKGROUND OF THE DISCLOSURE

Daprodustat, 2-[(1,3-dicyclohexyl-2,4,6-trioxo-1,3-diazinane-5-carbonyl)amino]acetic acid, has the following chemical structure:

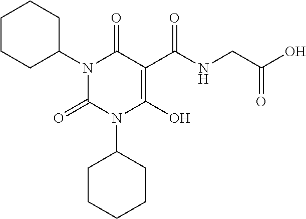 [SMILES: O=C(O)CNC(=O)c1c(O)n(C2CCCCC2)c(=O)n(C2CCCCC2)c1=O]

Daprodustat is a prolyl hydroxylase inhibitor, and it is developed for the treatment of anemia secondary to chronic kidney disease.

The compound is described in PCT publication WO 2007/150011.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis “TGA”, or differential scanning calorimetry “DSC”), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Daprodustat.

### SUMMARY OF THE DISCLOSURE

The present disclosure provides crystalline polymorphs of Daprodustat, processes for preparation thereof, and pharmaceutical compositions thereof. These crystalline polymorphs can be used to prepare other solid state forms of Daprodustat, Daprodustat salts and their solid state forms.

The present disclosure provides crystalline polymorphs of Daprodustat for use in the preparation of pharmaceutical compositions and/or formulations for use in medicine, preferably for the treatment of anemia secondary to chronic kidney disease.

The present disclosure also encompasses the use of crystalline polymorphs of Daprodustat of the present disclosure for the preparation of pharmaceutical compositions and/or formulations.

In another aspect, the present disclosure provides pharmaceutical compositions comprising crystalline polymorphs of Daprodustat according to the present disclosure.

In yet another embodiment, the present disclosure encompasses pharmaceutical formulations comprising the described any one or a combination of the crystalline polymorphs of Daprodustat, or pharmaceutical compositions comprising the described crystalline polymorphs of Daprodustat and at least one pharmaceutically acceptable excipient.

The present disclosure comprises processes for preparing the above mentioned pharmaceutical compositions. The processes comprise combining any one or a combination of the crystalline polymorphs of Daprodustat with at least one pharmaceutically acceptable excipient.

The crystalline polymorph of Daprodustat as defined herein and the pharmaceutical compositions or formulations of the crystalline polymorph of Daprodustat may be used as medicaments, particularly for the treatment of anemia secondary to chronic kidney disease.

The present disclosure also provides methods of treating anemia secondary to chronic kidney disease, comprising administering a therapeutically effective amount of any one or a combination of the crystalline polymorphs of Daprodustat of the present disclosure, or at least one of the above pharmaceutical compositions or formulations, to a subject suffering from anemia secondary to chronic kidney disease, or otherwise in need of the treatment.

The present disclosure also provides the uses of crystalline polymorphs of Daprodustat of the present disclosure, or at least one of the above pharmaceutical compositions or formulations, for the manufacture of medicaments for treating e.g.: anemia secondary to chronic kidney disease.

### BRIEF DESCRIPTION OF THE DRAWINGS

FIG. 1 shows a characteristic X-ray powder diffraction pattern (XRPD) of Daprodustat Form 1;

FIG. 2 shows a characteristic XRPD of a mixture of Daprodustat Form 1 and Form 2;

FIG. 3 shows a characteristic XRPD of Daprodustat Form 3 (prepared according to Example 3);

FIG. 4 shows a characteristic XRPD of Daprodustat Form 4;

FIG. 5 shows a characteristic XRPD of Daprodustat Form 3 (prepared according to Example 6);

FIG. 6 shows a characteristic DSC thermogram of Daprodustat Form 3;

FIG. 7 shows a characteristic DSC thermogram of Daprodustat Form 4;

FIG. 8 shows a characteristic TGA thermogram of Daprodustat Form 3;

FIG. 9 shows a characteristic TGA thermogram of Daprodustat Form 4;

FIG. 10a shows a characteristic solid state ¹³C NMR spectrum of Daprodustat Form 4;

FIG. 10b shows a characteristic solid state ¹³C NMR spectrum of Daprodustat Form 4 (at the range 0-100 ppm); and

FIG. 10c shows a characteristic solid state ¹³C NMR spectrum of Daprodustat Form 4 (at the range 100-200 ppm).

### DETAILED DESCRIPTION OF THE DISCLOSURE

The present disclosure encompasses solid state forms of Daprodustat, particularly crystalline polymorphs of Daprodustat, processes for preparation thereof, and pharmaceutical compositions thereof.

Solid state properties of Daprodustat and crystalline polymorphs thereof can be influenced by controlling the conditions under which Daprodustat and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Daprodustat described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Daprodustat. In some embodiments of the disclosure, the described crystalline polymorph of Daprodustat may contain from about 1% to about 20% (w/w), from about 5% to about 20% (w/w), or from about 5% to about 10% (w/w) of one or more other crystalline polymorph of the same Daprodustat.

Depending on which other crystalline polymorphs a comparison is made, the crystalline polymorphs of Daprodustat of the present disclosure has advantageous properties selected from at least one of the following: chemical purity, flowability, solubility, dissolution rate, morphology or crystal habit, stability—such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility, and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Daprodustat referred to herein as being characterized by graphical data “as depicted in” or “as substantially depicted in” a Figure will thus be understood to include any crystal forms of Daprodustat characterized with the graphical data having such small variations, as are well known to the skilled person, in comparison with the Figure.

As used herein, and unless stated otherwise, the term “anhydrous” in relation to crystalline forms of Daprodustat, relates to a crystalline form of Daprodustat which does not include any crystalline water (or other solvents) in a defined, stoichiometric amount within the crystal. Moreover, an “anhydrous” form would typically not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Daprodustat of the present disclosure corresponds to a crystalline polymorph of Daprodustat that is physically separated from the reaction mixture in which it is formed.

As used herein, unless stated otherwise, XRPD peaks reported herein are measured using CuKα radiation, λ=1.54184 Å. The XRPD measurements are typically carried out at a temperature of 25±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, typically about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

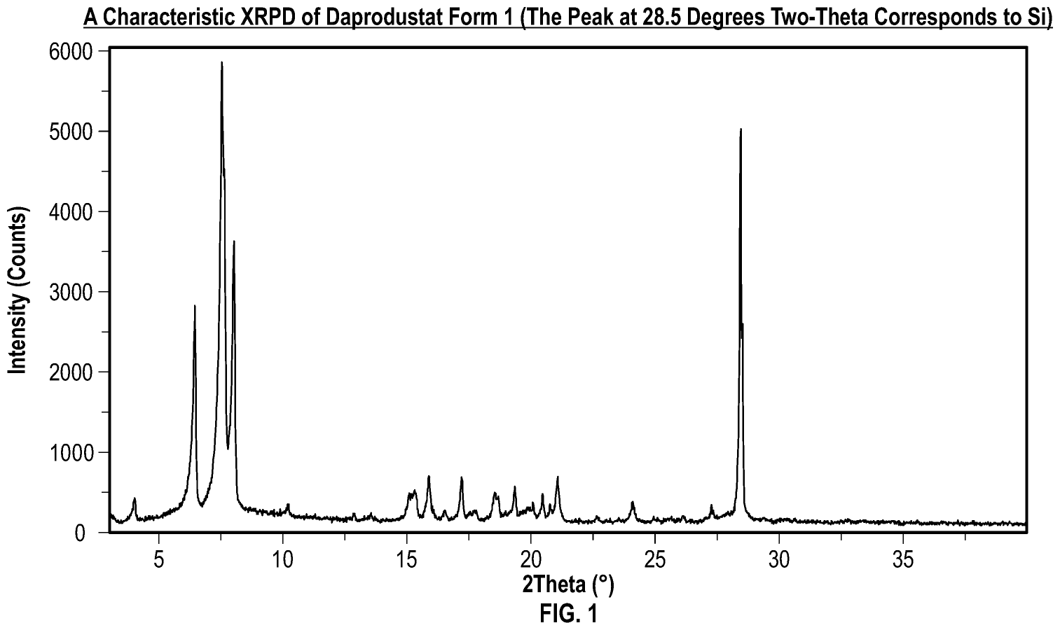

The present disclosure includes a crystalline polymorph of Daprodustat, designated Form 1. The crystalline Form 1 of Daprodustat may be characterized by data selected from one or more of the following: an X-ray powder diffraction pattern substantially as depicted in FIG. 1; an X-ray powder diffraction pattern having peaks at 6.4, 7.5, 15.9, 17.2 and 21.1 degrees 2-theta±0.2 degrees 2-theta; and combinations of these data.

Crystalline Form 1 of Daprodustat may be further characterized by an X-ray powder diffraction pattern having peaks at 6.4, 7.5, 15.9, 17.2 and 21.1 degrees 2-theta±0.2 degrees 2-theta, and also having any one, two, three, four or five additional peaks selected from 4.0, 7.6, 8.0, 19.3 and 24.1 degrees 2-theta±0.2 degrees 2-theta.

Crystalline Form 1 of Daprodustat may alternatively be characterized by an X-ray powder diffraction pattern having peaks at 4.0, 6.4, 7.5, 7.6, 8.0, 15.9, 17.2, 19.3, 21.1, and 24.1 degrees 2-theta±0.2 degrees 2-theta.

Crystalline Form 1 of Daprodustat may be characterized by each of the above characteristics alone/or by all possible combinations, e.g., an XRPD pattern having peaks at 6.4, 7.5, 15.9, 17.2 and 21.1 degrees 2-theta±0.2 degrees 2-theta; an XRPD pattern as depicted in FIG. 1, and combinations thereof.

In one embodiment of the present disclosure, crystalline Form 1 of Daprodustat is isolated.

Crystalline Form 1 of Daprodustat may be anhydrous form.

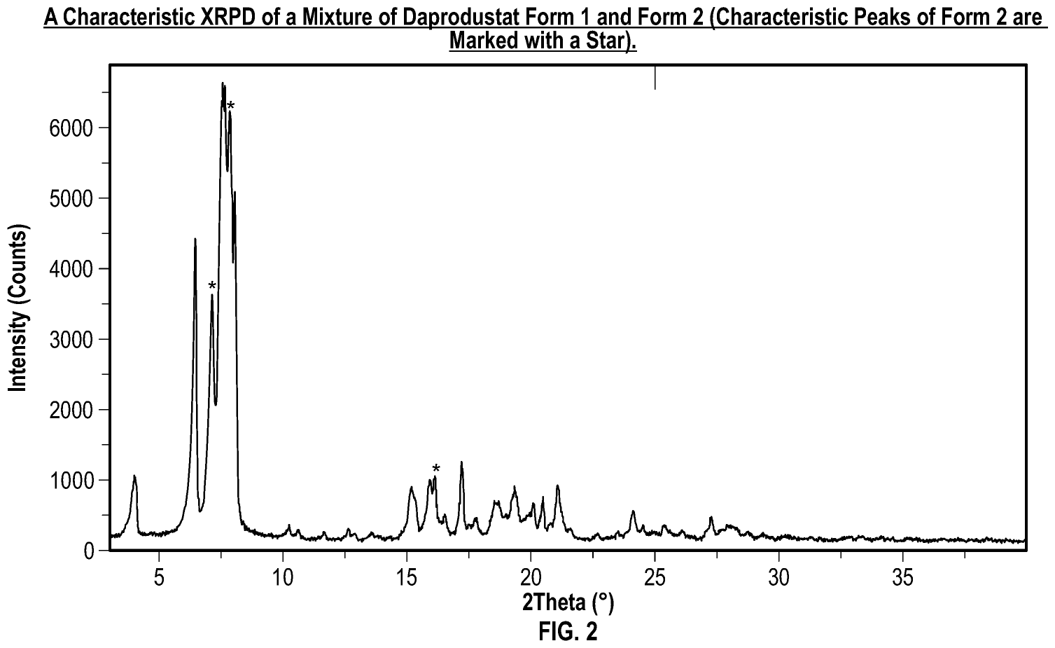

The present disclosure further includes a mixture of Daprodustat crystalline Form 1 and additional crystalline form, designated Form 2. The mixture can be characterized by an X-ray powder diffraction pattern substantially as depicted in FIG. 2.

The present disclosure further includes a crystalline polymorph of Daprodustat, designated Form 2. The crystalline Form 2 of Daprodustat may be characterized by an X-ray powder diffraction pattern having peaks at 7.1, 7.8 and 16.1 degrees 2-theta±0.2 degrees 2-theta; and combinations of these data.

Crystalline Form 2 of Daprodustat may be anhydrous form.

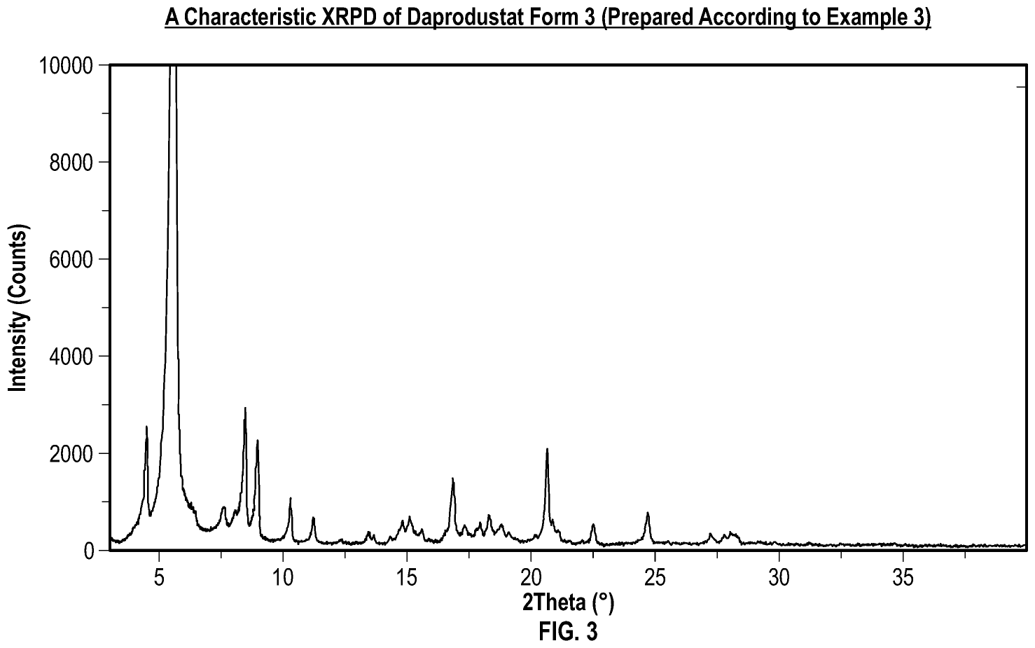

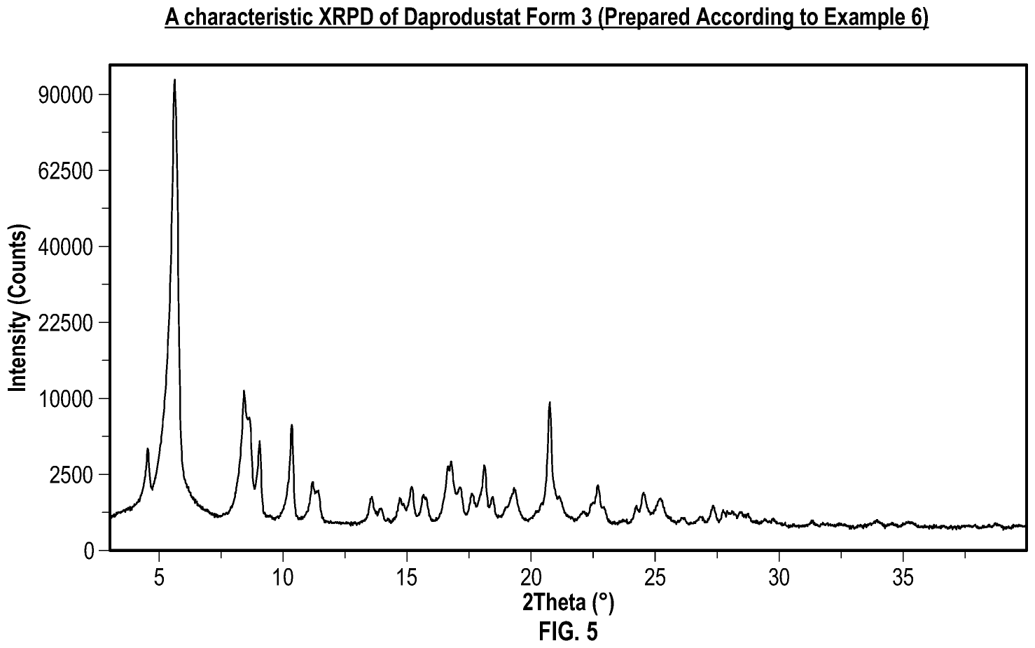

The present disclosure also includes a crystalline polymorph of Daprodustat, designated Form 3. The crystalline Form 3 of Daprodustat may be characterized by data selected from one or more of the following: an X-ray powder diffraction pattern substantially as depicted in FIG. 3 or in FIG. 5; an X-ray powder diffraction pattern having peaks at 4.5, 5.6, 9.0 and 16.8 degrees 2-theta±0.2 degrees 2-theta; an X-ray powder diffraction pattern having peaks at 4.5, 5.6, 6.7, 9.0 and 16.8 degrees 2-theta±0.2 degrees 2-theta; and combinations of these data.

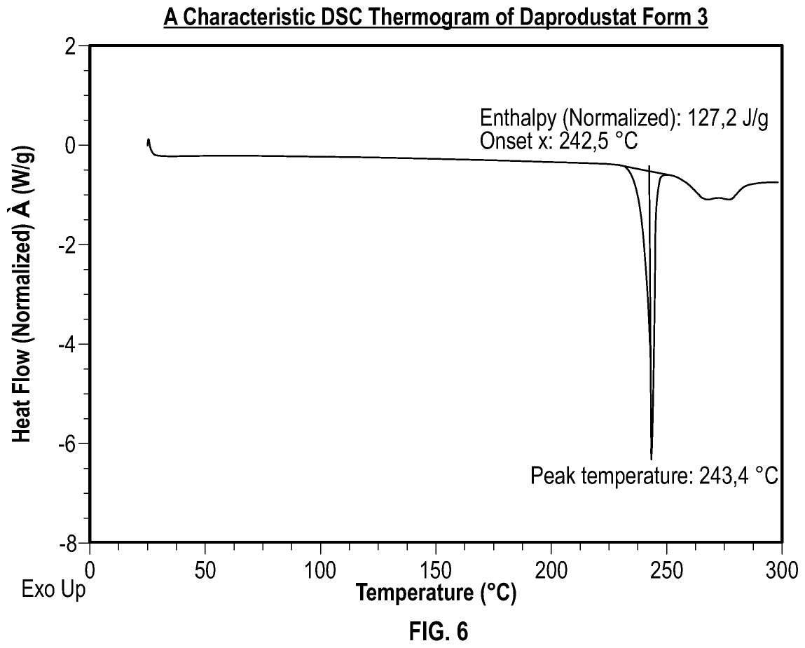

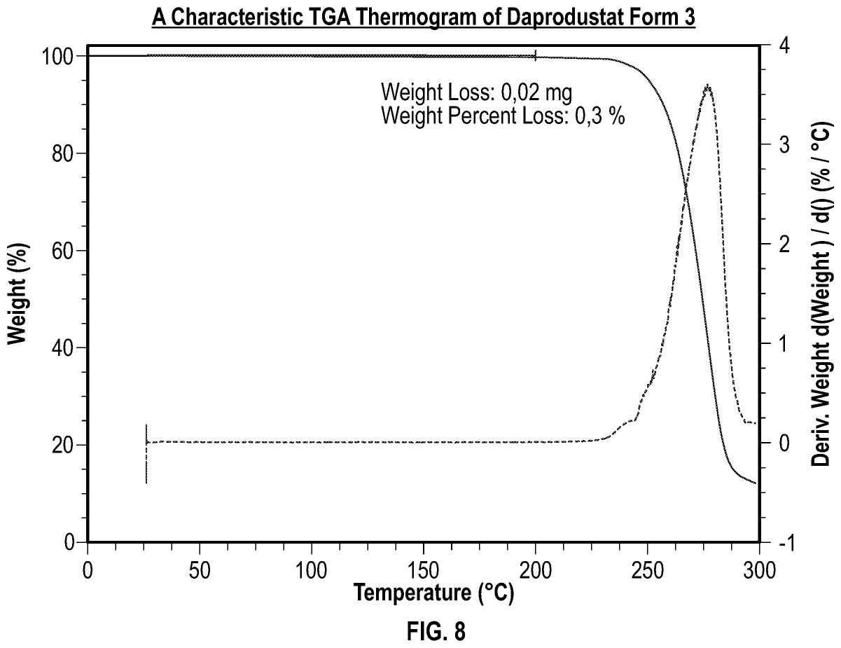

Crystalline Form 3 of Daprodustat may be further characterized by data selected from one or more of the following: an X-ray powder diffraction pattern having peaks at 4.5, 5.6, 9.0 and 16.8 degrees 2-theta±0.2 degrees 2-theta, and also having any one, two, three, four or five additional peaks selected from 8.5, 11.2, 20.6 and 24.7 degrees 2-theta±0.2 degrees 2-theta; a DSC endothermic peak with T onset at about 245.3° C.; a DSC thermogram as depicted in FIG. 6; A TGA thermogram as depicted in FIG. 8; and combinations of these data.

Crystalline Form 3 of Daprodustat may be alternatively characterized by an X-ray powder diffraction pattern having peaks at 4.5, 5.6, 8.5, 9.0, 11.2, 16.8, 20.6 and 24.7 degrees 2-theta±0.2 degrees 2-theta.

Crystalline Form 3 of Daprodustat may be characterized by each of the above characteristics alone/or by all possible combinations, e.g., an XRPD pattern having peaks at 4.5, 5.6, 9.0 and 16.8 degrees 2-theta±0.2 degrees 2-theta; an XRPD pattern as depicted in FIG. 3 or in FIG. 5, and combinations thereof.

In one embodiment of the present disclosure, crystalline Form 3 of Daprodustat is isolated.

Crystalline Form 3 of Daprodustat may be anhydrous form; typically Form 3 has weight loss of about 0.34% (w/w) as measured by TGA.

Crystalline Form 3 of Daprodustat may have advantageous properties, as described above. Particularly, Crystalline Form 3 is polymorphically and chemically stable. TGA analysis shows that Form 3 has a high Tₒₙₛₑₜ which correlates with a thermodynamically stable form. For example, Form 3 may be stored in closed bottles for at least 2 weeks.

Crystalline Form 3 of Daprodustat may be prepared by a process comprising crystallizing Daprodustat from ethylbenzene. In an embodiment, the process comprises:
a. preparing a solution of Daprodustat in ethylbenzene at a temperature of about 40° C. to about 100° C.; and
b. maintaining the solution at a temperature of about 10° C. to about 35° C. to obtain a solid; and optionally isolating the solid.

Preferably, the temperature in step (a) is about 50° C. to about 80° C., and more preferably about 55° C. to about 70° C. Preferably, the ethylbenzene is present in a ratio of about 50 ml to about 300 ml per gram of Daprodustat, more preferably about 80 ml to about 200 ml per gram of Daprodustat, and particularly about 90 ml to about 110 ml per gram of Daprodustat, or about 100 ml per gram of Daprodustat.

Step (b) preferably comprises maintaining the solution at a temperature of about 15° C. to about 30° C. or about 20° C. to 25° C., or at room temperature. The solution in step (a) is preferably allowed to cool to this temperature. Preferably the solution is maintained at this temperature range for about 8 hours to about 72 hours, about 10 hours to about 60 hours, or about 12 to about 36 hours and particularly about 24 hours.

The product may be isolated by filtration. The isolated product can be dried, typically at a temperature of not more than about 40° C., for a period of about up to 2 hours.

Crystalline Form 3 of Daprodustat may also be prepared by a process comprising crystallizing Daprodustat from a mixture of methyl-tetrahydrofuran and n-heptane. The process may comprise:
a. preparing a solution of Daprodustat in methyl-tetrahydrofuran;
b. preparing a suspension of crystalline Form 3 of Daprodustat in n-heptane;
c. combining the solution of Daprodustat in methyl-tetrahydrofuran of step a) with the suspension of crystalline Form 3 of Daprodustat in n-heptane of step b), to obtain a suspension; and
d. isolating crystalline Form 3 of Daprodustat.

Preferably, the temperature in step (a) is about 15° C. to about 40° C., about 18° C. to about 30° C., and more preferably about 20° C. to about 25° C., and particularly at room temperature. Preferably, the methyl-tetrahydrofuran is present in a ratio of about 30 ml to about 200 ml per gram of Daprodustat in step (a), more preferably about 50 ml to about 150 ml per gram of Daprodustat, and particularly about 60 ml to about 80 ml per gram of Daprodustat in step (a), or about 70 ml per gram of Daprodustat in step (a). The solution in step (a) may be filtered prior to step (c).

Preferably, the ratio of THF to n-heptane in step (b) may be about 1:1 to about 1:8; about 1:2 to about 1:6, or about 1:4. Preferably, the n-heptane is present in a ratio of about 30 ml to about 200 ml per gram of Daprodustat in step (b), more preferably about 100 ml to about 400 ml per gram of Daprodustat in step (b), and particularly about 200 ml to about 350 ml per gram of Daprodustat step (b), or about 280 ml per gram of Daprodustat in step (b).

Preferably, the temperature in step (b) is about −5° C. to about 20° C., more preferably about −2° C. to about 10° C. and particularly about 0° C. to about 5° C.

Preferably, step (c) comprises addition (e.g. dropwise addition) of the solution of Daprodustat in methyl-tetrahydrofuran to the suspension of Daprodustat Form 3 in n-heptane. Preferably the mixture in step (c) is maintained at a temperature of about −5° C. to about 20° C., more preferably about −2° C. to about 10° C. and particularly about 0° C. to about 5° C.

The solid is preferably recovered by filtration and washed, preferably with n-heptane. The resulting product is preferably dried, preferably at a temperature of about 25° C. to about 60° C., and more preferably about 30° C. to about 50° C., or about 40° C.

Crystalline Form 3 of Daprodustat may also be prepared by a process comprising crystallizing Daprodustat from p-xylene. In an embodiment, the process comprises:
a. preparing a solution of Daprodustat in p-xylene, preferably at a temperature of about 80° C. to about 150° C.; and
b. maintaining the solution at a temperature of about 10° C. to about 35° C. to obtain a solid; and optionally isolating the solid.

Preferably, the temperature in step (a) is about 100° C. to about 145° C., and more preferably about 130° C. to about 140° C., or about 138° C. Preferably, the p-xylene is present in a ratio of about 40 ml to about 200 ml per gram of Daprodustat, more preferably about 60 ml to about 120 ml per gram of Daprodustat, and particularly about 70 ml to about 100 ml per gram of Daprodustat, or about 86 ml per gram of Daprodustat.

Step (b) preferably comprises maintaining the solution at a temperature of about 15° C. to about 30° C. or about 20° C. to 25° C., or at room temperature. The solution in step (a) is preferably allowed to cool to this temperature. Preferably the solution is maintained at this temperature range for about 8 hours to about 72 hours, about 10 hours to about 60 hours, or about 12 to about 36 hours and particularly about 24 hours.

The product may be isolated by filtration. The isolated product may be dried, typically at a temperature of not more than about 40° C., for a period of about up to 2 hours.

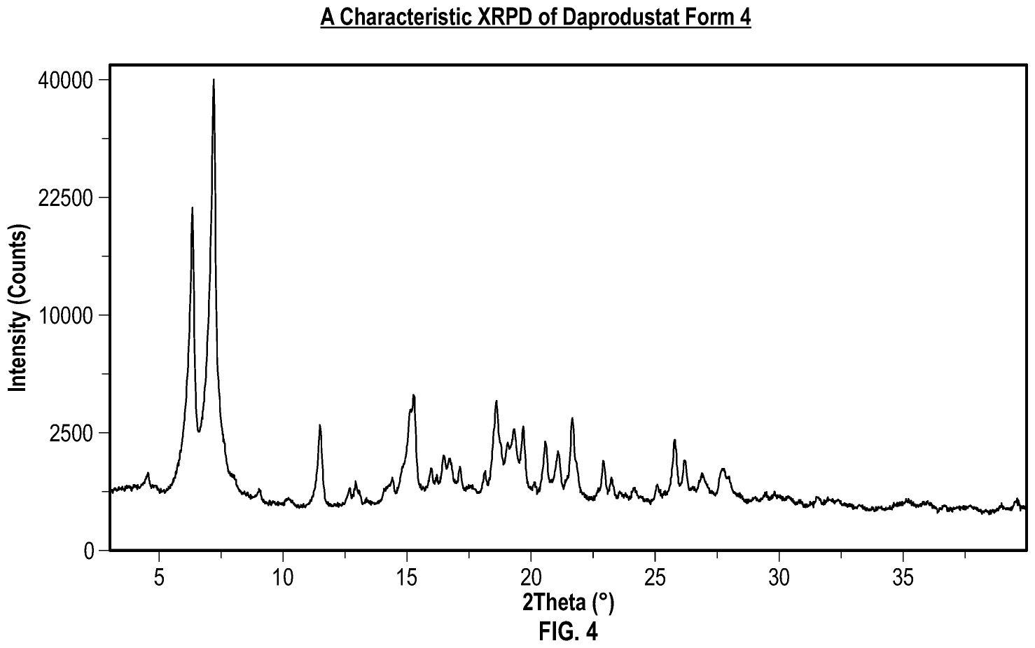

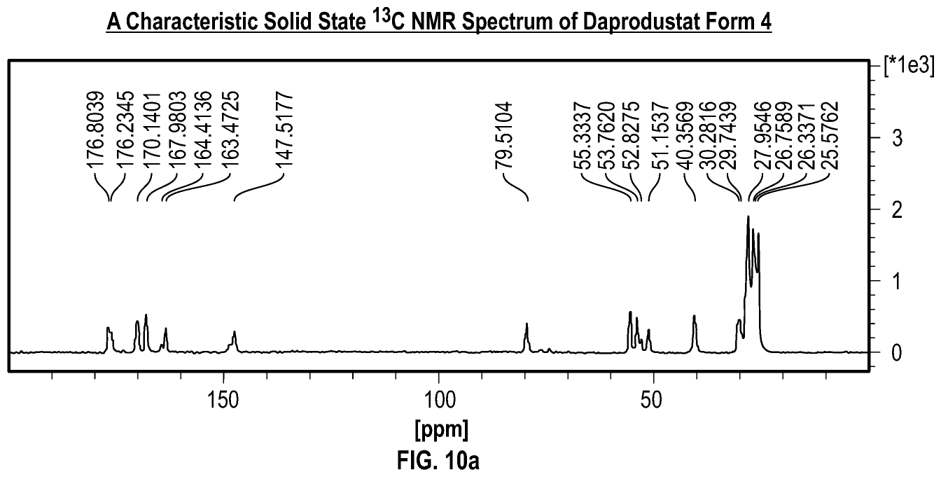

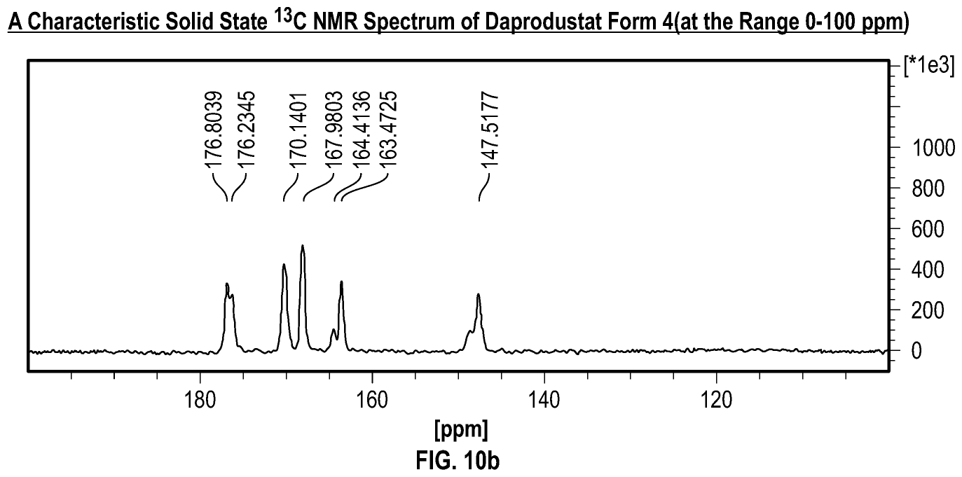

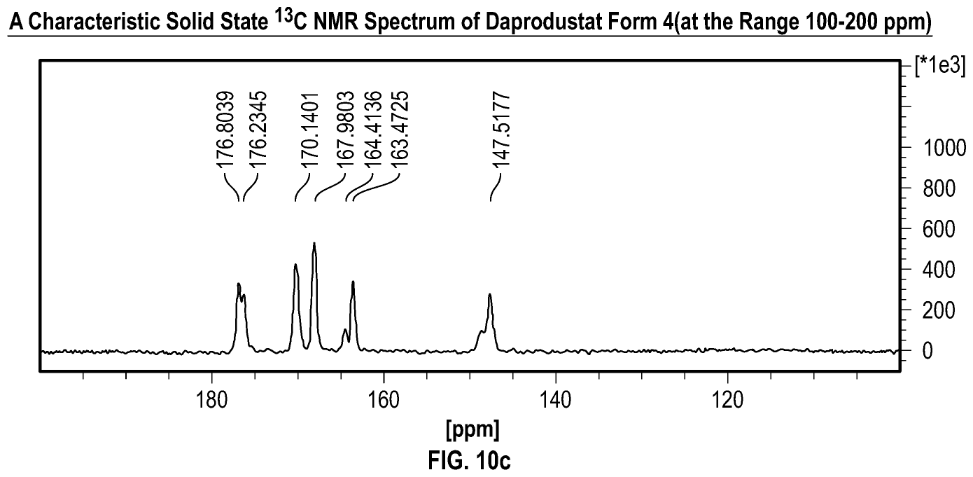

The present disclosure also includes a crystalline polymorph of Daprodustat, designated Form 4. The crystalline Form 4 of Daprodustat may be characterized by data selected from one or more of the following: an X-ray powder diffraction pattern substantially as depicted in FIG. 4; an X-ray powder diffraction pattern having peaks 7.2, 11.5, 21.7, 22.9, 23.3 and 25.8 degrees 2-theta±0.2 degrees 2-theta; a solid state ¹³C NMR spectrum substantially as depicted in either FIG. 10a, 10b or 10c; and combinations of these data.

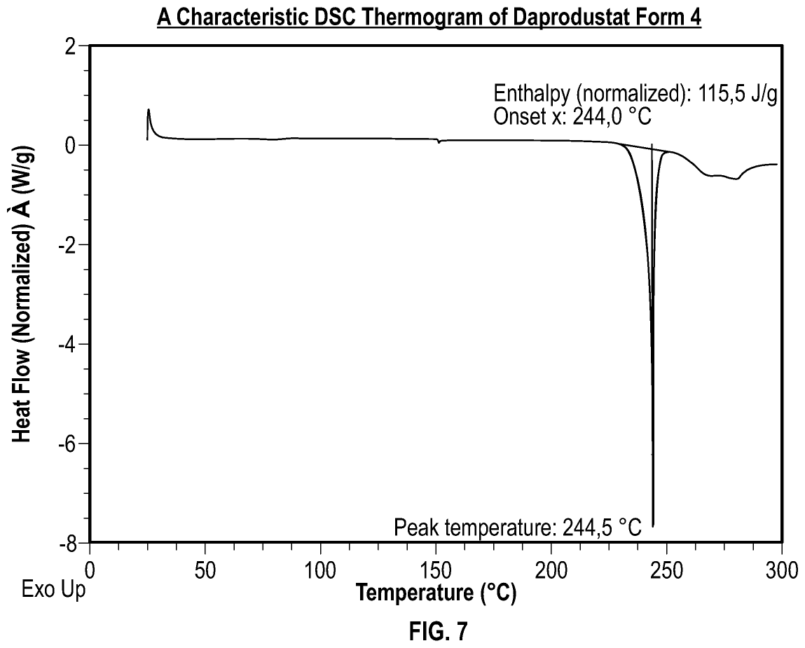

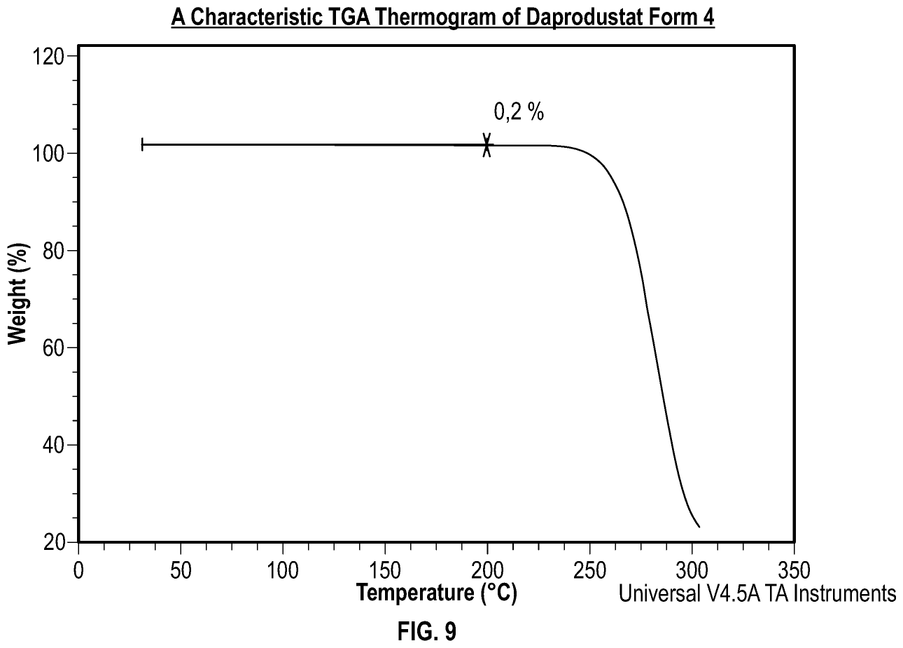

Crystalline Form 4 of Daprodustat may be further characterized by data selected from one or more of the following: an X-ray powder diffraction pattern having peaks at 7.2, 11.5, 21.7, 22.9, 23.3 and 25.8 degrees 2-theta±0.2 degrees 2-theta, and also having any one, two, three, four or five additional peaks selected from 6.3, 12.9, 16.5, 18.1 and 19.7 degrees 2-theta±0.2 degrees 2-theta; a DSC endothermic peak with T onset at about 243.9° C.; a DSC thermogram as depicted in FIG. 7; a TGA thermogram as depicted in FIG. 9; and combinations of these data.

Crystalline Form 4 of Daprodustat may be alternatively characterized by an X-ray powder diffraction pattern having peaks at 6.3, 7.2, 11.5, 12.9, 16.5, 18.1, 19.7, 21.7, 22.9, 23.3 and 25.8 degrees 2-theta±0.2 degrees 2-theta.

Crystalline Form 4 of Daprodustat may be characterized by each of the above characteristics alone/or by all possible combinations, e.g., an XRPD pattern having peaks at 7.2, 11.5, 21.7, 22.9, 23.3 and 25.8 degrees 2-theta±0.2 degrees 2-theta; an XRPD pattern as depicted in FIG. 4, and combinations thereof.

In one embodiment of the present disclosure, crystalline Form 4 of Daprodustat is isolated.

Crystalline Form 4 of Daprodustat may be an anhydrous form; typically Form 4 has weight loss of about 0.2% (w/w) as measured by TGA.

Crystalline Form 4 of Daprodustat may be characterized by each of the above characteristics alone or by all possible combinations, e.g., an XRPD pattern having peaks at 7.2, 11.5, 21.7, 22.9, 23.3 and 25.8 degrees 2-theta±0.2 degrees 2-theta; an XRPD pattern as depicted in FIG. 4, and combinations thereof.

Crystalline Form 4 of Daprodustat may be prepared by a process comprising slurrying crystalline Form 1 of Daprodustat in diethyl ether. The slurrying may be carried out at a temperature of about −5° C. to about 30° C., preferably −5° C. to about 20° C., about −5° C. to about 5° C., or about 0° C. The diethyl ether is present in a ratio of about 2 ml to about 40 ml per gram of Daprodustat, preferably about 5 ml to about 20 ml per gram of Daprodustat, or about 8 ml to about 15 ml per gram of Daprodustat, or about 10 ml per gram of Daprodustat. Typically, the slurrying may be carried out or a sufficient amount of time to prepare Form 4, particularly about 20 hours to about 120 hours, about 36 hours to about 90 hours, about 36 hours to about 80 hours, or about 50 hours to about 70 hours.

The above crystalline polymorphs can be used to prepare other crystalline polymorphs of Daprodustat, Daprodustat salts and their solid state forms.

The present disclosure provides the above described crystalline polymorphs of Daprodustat for use in the preparation of pharmaceutical compositions comprising Daprodustat and/or crystalline polymorphs thereof.

The present disclosure also encompasses the use of crystalline polymorphs of Daprodustat of the present disclosure for the preparation of pharmaceutical compositions of crystalline polymorph Daprodustat and/or crystalline polymorphs thereof.

The present disclosure comprises processes for preparing the above mentioned pharmaceutical compositions. The processes comprise combining any one or a combination of the crystalline polymorphs of Daprodustat of the present disclosure with at least one pharmaceutically acceptable excipient.

Pharmaceutical formulations of the present invention contain any one or a combination of the solid state forms of Daprodustat of the present invention. In addition to the active ingredient, the pharmaceutical formulations of the present invention can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Primelloseg), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Daprodustat and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present invention, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

A pharmaceutical formulation of Daprodustat can be administered. Daprodustat is preferably formulated for administration to a mammal, preferably a human, by injection. Daprodustat can be formulated, for example, as a viscous liquid solution or suspension, preferably a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Daprodustat and the pharmaceutical compositions and/or formulations of Daprodustat of the present disclosure can be used as medicaments, particularly in the treatment of anemia secondary to chronic kidney disease.

The present disclosure also provides methods of treating anemia secondary to chronic kidney disease comprising administering a therapeutically effective amount of any one or a combination of the crystalline polymorphs of Daprodustat of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

The sample was powdered in a mortar and pestle and applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 s, continuous scan.

The described peak positions were determined with or without using silicon powder as an internal standard in an admixture with the sample measured. In some measurements, the described peak positions were determined using silicon powder as an internal standard in an admixture with the sample measured. The position of the silicon (Si) peak was corrected to silicone theoretical peak: 28.45 degrees two theta, and the positions of the measured peaks were corrected respectively.

### Differential Scanning Calorimetry (“DSC”) Method

DSC measurements were done using TA Instruments Discovery, DSC unit. 1-3 mg of sample was weighted in pan, hermetically closed with the pin hole. Sample was purged with 50 ml/min of nitrogen flow and heated in the range of 25-300° C., with heating rate of 20° C./min.

### Thermo Gravimetric Analysis (“TGA”)

TGA measurements were done using TA Instruments Discovery, TG unit. 5-10 mg of sample was weighted in open aluminum pan. Sample was purged with 50 ml/min of nitrogen flow and heated in the range of 25-300° C., with heating rate of 10° C./min.

Solid-State Nucleic Magnetic Resonance (“ss-NMR”)

Solid-state 13C NMR spectra were recorded with variable amplitude cross polarization, magic angle spinning and high power proton decoupling using a BRUKER Avance II+ spectrometer operating at 125.77 MHz and 303 K. A probe using 4 mm o.d. zirconia rotors was employed. The operation conditions were: contact time: 1 ms; recycle delay: 5 s; 2048 scans and spin rate of 18 kHz. Chemical shifts were referenced via a replacement sample of glycine (carboxyl carbon chemical shift assigned as 176.03 ppm relative to the signal of tetramethylsilane).

### Preparation of Starting Materials

Daprodustat can be prepared according to methods known from the literature, for example International Publication No. WO 2007/150011 (corresponding to U.S. Pat. No. 8,324,208).

### Example 1. Preparation of Daprodustat Form 1

Crude Daprodustat (30 mg) was dissolved in acetone (2 mL) at temperature of about 50° C. and a clear solution was formed. The clear solution was left at room temperature for 1 day to crystallize. The obtained solid was filtered under vacuum. The obtained solid was analyzed by XRPD, form 1 was obtained. An XRPD pattern is shown in FIG. 1.

### Example 2. Preparation of Form 2 in a Mixture with Form 1

Crude Daprodustat (30 mg) was dissolved in Methanol (3 mL) at temperature of about 60° C. and a clear solution was formed. The clear solution was left at room temperature for 3 days to crystallize. The obtained solid was filtered. The obtained solid was analyzed by XRPD, a mixture of form 1 and form 2 was obtained. An XRPD pattern is shown in FIG. 2.

### Example 3. Preparation of Daprodustat Form 3

Crude Daprodustat (30 mg) was dissolved in ethylbenzene (3 mL) at temperature of about 60° C. and a clear solution was formed. The clear solution was left at room temperature for 1 day to crystallize. The obtained solid was filtered under vacuum. The obtained solid was analyzed by XRPD, form 3 was obtained. An XRPD pattern is shown in FIG. 3.

### Example 4. Preparation of Daprodustat Form 4

Daprodustat (100 mg, Form 1) was slurried in Diethyl ether (1 mL) at about 0° C. for 66 hours. The obtained solid was filtered and analyzed by XRPD. Form 4 was obtained. An XRPD pattern is shown in FIG. 4.

### Example 5. Preparation of Daprodustat Form 3

Daprodustat (100 mg, crude) was dissolved in methyl-tetrahydrofuran (7 ml) at room temperature. The solution was stirred for about 1 hour and then it was filtered. Form 3 (10 mg) was suspended in n-heptane (28 ml) at a temperature of about 0-5° C. 1.5 ml of the solution of Daprodustat in methyl tetrahydrofuran was added dropwise into the suspension of Form 3 in n-heptane, and the obtained mixture was stirred for 10 minutes at a temperature of about 0-5° C. Then, the rest of the solution was added dropwise and the obtained suspension was filtered and the material was washed with n-heptane (8 ml). The isolated material was dried for 2 hours at a temperature of 40° C., and was analyzed by XRPD. Form 3 was obtained. The sample was also analysed by TGA:weight loss of about 0.34% (w/w).

### Example 6. Preparation of Daprodustat Form 3

Crude Daprodustat (35 mg) was dissolved in p-Xylene (3 mL) at temperature of about 138° C. and a clear solution was formed. The clear solution was left at room temperature for 1 day to crystallize. The obtained solid was filtered under vacuum. The obtained solid was analyzed by XRPD. Form 3 was obtained. An XRPD pattern is shown in FIG. 5.

1. Crystalline Form 3 of Daprodustat, characterized by data selected from one or more of the following:
a. an X-ray powder diffraction pattern substantially as depicted in FIG. 3 or in FIG. 5;
b. an X-ray powder diffraction pattern having peaks at 4.5, 5.6, 9.0 and 16.8 degrees 2-theta±0.2 degrees 2-theta; and
c. combinations of these data.

2. Crystalline Form 3 of Daprodustat according to claim 1, characterized by data selected from one or more of the following:
a. an X-ray powder diffraction pattern having peaks at 4.5, 5.6, 9.0 and 16.8 degrees 2-theta±0.2 degrees 2-theta, and also having any one, two, three, four or five additional peaks selected from 8.5, 11.2, 20.6 and 24.7 degrees 2-theta±0.2 degrees 2-theta;
b. a DSC endothermic peak with T onset at about 245.3° C.;
c. a DSC thermogram as depicted in FIG. 6;
d. a TGA thermogram as depicted in FIG. 8; and
e. combinations of these data.

3. Crystalline Form 3 of Daprodustat according to claim 1, wherein the crystalline form is an anhydrous form.

4. A process for preparing crystalline Form 3 of Daprodustat, comprising crystallizing Daprodustat from ethylbenzene.

5. A process according to claim 4, comprising
a. dissolving Daprodustat in ethylbenzene at a temperature of about 40° C. to about 100° C. to obtain a solution; and
b. maintaining the solution at a temperature of about 10° C. to about 35° C. to obtain a solid; and optionally isolating crystalline Form 3 of Daprodustat.

6. A process for preparing crystalline Form 3 of Daprodustat, comprising crystallizing Daprodustat from a mixture of methyl-tetrahydrofuran and n-heptane.

7. A process according to claim 6, comprising
a. preparing a solution of Daprodustat in methyl-tetrahydrofuran;
b. preparing a suspension of crystalline Form 3 of Daprodustat in n-heptane;
c. combining the solution in step (a) and the suspension in step (b) to obtain a suspension; and optionally
d. isolating crystalline Form 3 of Daprodustat.

8. A process for preparing crystalline Form 3 of Daprodustat, comprising crystallizing Daprodustat from p-xylene.

9. A process according to claim 8, comprising
a. dissolving Daprodustat in p-xylene at a temperature of about 80° C. to about 150° C. obtain a solution; and
b. maintaining the solution at a temperature of about 10° C. to about 35° C. to obtain a solid; and optionally
c. isolating crystalline Form 3 of Daprodustat.

10. Crystalline Form 4 of Daprodustat, characterized by data selected from one or more of the following:
a. an X-ray powder diffraction pattern substantially as depicted in FIG. 4;
b. an X-ray powder diffraction pattern having peaks 7.2, 11.5, 21.7, 22.9, 23.3 and 25.8 degrees 2-theta±0.2 degrees 2-theta;
c. a solid state ¹³C NMR spectrum substantially as depicted in a solid state ¹³C NMR spectrum substantially as depicted in any one of FIGS. 10a, 10b and 10c; and
d. combinations of these data.

11. Crystalline Form 4 of Daprodustat according to claim 10, characterized by data selected from one or more of the following:
a. an X-ray powder diffraction pattern having peaks at 7.2, 11.5, 21.7, 22.9, 23.3 and 25.8 degrees 2-theta±0.2 degrees 2-theta, and also having any one, two, three, four or five additional peaks selected from 6.3, 12.9, 16.5, 18.1 and 19.7 degrees 2-theta±0.2 degrees 2-theta;
b. a DSC endothermic peak with T onset at about 243.9° C.;
c. a DSC thermogram as depicted in FIG. 7;
d. a TGA thermogram as depicted in FIG. 9; and
e. combinations of these data.

12. Crystalline Form 4 of Daprodustat according to claim 10, wherein the crystalline form is an anhydrous form.

13. A process for preparing crystalline Form 4 of Daprodustat, comprising slurrying crystalline Form 1 of Daprodustat in diethyl ether.

14. A process according to claim 13, wherein the slurrying is performed for a period of from about 20 hours to about 120 hours.

15. A process according to claim 4, comprising isolating the crystalline Daprodustat and combining the crystalline Daprodustat with at least one pharmaceutically acceptable excipient to prepare a pharmaceutical formulation.

16. Crystalline Daprodustat obtainable by a process according to claim 4.

17. Crystalline Daprodustat according to claim 1, which contains: no more than about 20% of any other crystalline forms of Daprodustat.

18. Crystalline Daprodustat according to claim 1, which contains no more than about 20% of amorphous Daprodustat.

19. Crystalline Daprodustat according to claim 16, which contains no more than about 20% of other solid state forms of Daprodustat.

20. A pharmaceutical composition comprising the crystalline Daprodustat according to claim 1.

21. (canceled)

22. A pharmaceutical formulation comprising the crystalline Daprodustat according to claim 1, and at least one pharmaceutically acceptable excipient.

23. A process for preparing the pharmaceutical formulation according to claim 22, comprising combining the crystalline Daprodustat with at least one pharmaceutically acceptable excipient.

24. (canceled)

25. (canceled)

26. A method of treating anemia secondary to chronic kidney disease, comprising administering a therapeutically effective amount of the crystalline Daprodustat according to claim 1 to a subject in need of treatment.

27. (canceled)

28. (canceled)

29. (canceled)
